(19) United States
(12) Patent Application Publication
(10) Pub. No.: US 2014/0014110 A1
(43) Pub. Date: Jan. 16, 2014

(54) REMOTELY CONTROLLED POSITIVE AIRWAY-PRESSURE APPARATUS AND METHOD

(71) Applicant: Phillip M. Adams, Afton, WY (US)

(72) Inventor: Phillip M. Adams, Afton, WY (US)

(21) Appl. No.: 13/549,691

(22) Filed: Jul. 16, 2012

(51) Int. Cl. A61M 16/06

(52) U.S. Cl. 128/204.21

(57) ABSTRACT

A CPAP system and method may include a base comprising a flow generator, a respiratory interface, and an elongated flexible conduit extending to connect the base to the respiratory interface. The CPAP system may further include a remote user interface controlling at least certain aspects of the flow generator. For example, the remote user interface may selectively transition, in accordance with commands input therethrough, the flow generator between an active, “on” condition and an inactive, “off” condition. The remote user interface may be secured to move with a respirator interface. Alternatively, the remote user interface may be secured at a location of the user's choosing.

### BACKGROUND

1. The Field of the Invention

This invention relates to treatment of sleep apnea and, more particularly, to novel systems and methods for controlling positive airway pressure (PAP) or similar positive airway pressure (PAP, BPAP, VPAP, IPAP, EPAP, APAP, CPAP etc) treatment systems.

2. The Background Art

Due to the number of various Positive Airway Pressure (PAP, BPAP, VPAP, IPAP, EPAP, APAP, CPAP etc) alternatives, and the fact that all are often grouped, somewhat inaccurately, as CPAP devices. the term CPAP or PAP used throughout this patent application should be interpreted as including any or all of these PAP alternatives.

Positive airway pressure (PAP) therapy is often used to treat obstructive sleep apnea as well as certain other disorders. In a PAP apparatus and method, pressurized air is delivered through a mask to a patient's airway. This is typically done while the user sleeps. Accordingly, such systems are set on a night stand or other support beside a bed, and operate from wall current or a battery power source. Typically, a fan in an air source called the “generator” blows ambient air to create a pressurized supply having a pressure head of from about five to fifteen centimeters of water. The mask or interface portion of the apparatus may be oral, oral-nasal, or simply nasal in its introduction of air.

Given the conditions and environment in which they are used, current CPAP systems are not particularly user friendly. For example, current CPAP systems do not lend themselves to a quick easy control response nor manipulation in a dark environment. Awaking from sleep to answer a phone is often unsuccessful due to an inability to find, reach, and timely shut off the noisy generator and flow and remove the mask. Accordingly, what is needed is a system and method that increases the efficiency and ease with which a user may interact with his or her CPAP system.

### BRIEF SUMMARY OF THE INVENTION

In view of the foregoing, in accordance with the invention as embodied and broadly described herein, a method and apparatus are disclosed in one embodiment of the present invention as including a CPAP treatment system. Such a system may provide treatment as a user sleeps and address certain challenges associated with operating in that environment.

For example, when a user is reclined in bed, a base or flow generating portion of a CPAP system may not be within reach or easy reach. Additionally, many users require corrective lenses, which may be removed as he or she readies for bed. Accordingly, a user may be required to find, reach, and manipulate a base (e.g., turn a flow generator on or off) with impaired vision. Furthermore, a user may be required to manipulate a base in poor lighting conditions and often, while not fully awake or alert.

In view of these and related challenges, a CPAP system in accordance with the present invention may include a remote user interface (i.e., a user interface that is spaced away from a base). In selected embodiments, a remote user interface may provide a mechanism through which a user may issue commands (e.g., commands to turn a flow generator on or off).

A remote user interface may comprise a unit separable from a mask, air hose, or the like. Such a remote interface may be secured or positioned at any location desired by a user (e.g., on a headboard, wall, wrist, headgear, or the like). Alternatively, a remote user interface may be integrated within (e.g., be built into) or otherwise secured to a mask itself or some hardware moving therewith. In either case, a remote user interface may be positioned to enable a user to quickly and repeatedly locate it. The user may do this in the dark by muscle memory or touch alone. Moreover, the user need not sit up, reach for, or locate a base to control its operation.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a perspective view of one embodiment of a remotely controlled CPAP system in accordance with the present invention;

FIG. 2 is a schematic block diagram of another embodiment of a remotely controlled CPAP system in accordance with the present invention, the CPAP system having a remote user interface incorporated within a remote (e.g., remote element or remote control device);

FIG. 3 is a schematic block diagram of another embodiment of a remotely controlled CPAP system in accordance with the present invention, the CPAP system having a remote user interface configured as a separate element or component;

FIG. 4 is a schematic block diagram of one embodiment of a delivery system for conducting air from a base to a respiratory interface in accordance with the present invention, the delivery system incorporating a communication link therewithin;

FIG. 5 is a schematic block diagram of another embodiment of a delivery system for conducting air from a base to a respiratory interface in accordance with the present invention, the delivery system supporting, but not incorporating, a communication link;

FIG. 6 is a schematic block diagram of another embodiment of a remotely controlled CPAP system in accordance with the present invention, the CPAP system comprising an upgrade enabling a remote user interface to exert a degree of control over a non-communicative CPAP base;

FIG. 7 is a perspective view of one embodiment of a controller of an upgrade in accordance with the present invention;

FIG. 8 is a perspective view of one embodiment of a combined remote user interface and communication link in accordance with the present invention;

FIG. 9 is a perspective view of one embodiment of a remote user interface configured as a separate unit contained within a small, unobtrusive housing in accordance with the present invention;

FIG. 10 is a perspective view of one embodiment of a remote user interface configured as an adapter or insert that may interface between a coupler of a delivery system and a remote in accordance with the present invention; and

FIG. 11 is a front elevation view of one embodiment of a remote showing various locations wherein a remote user interface or the input devices thereof may be located in accordance with the present invention.

### DETAILED DESCRIPTION OF SELECTED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

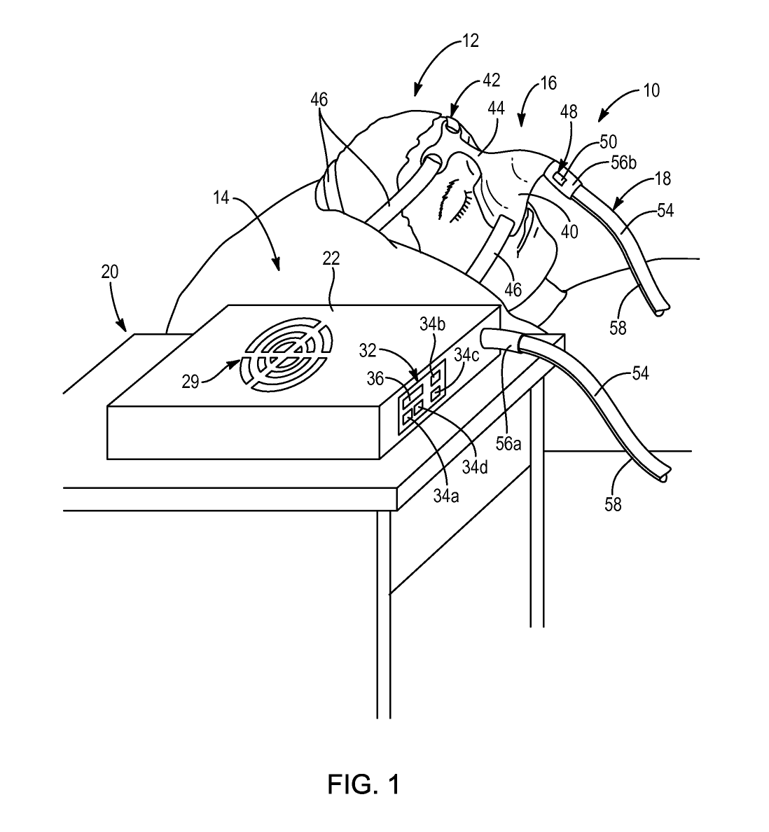

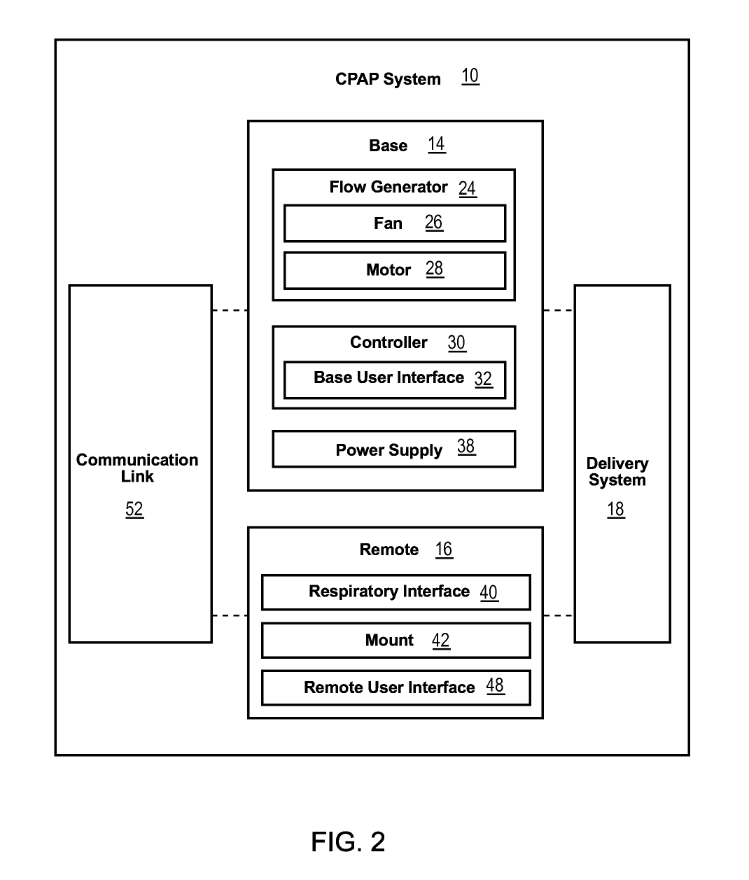

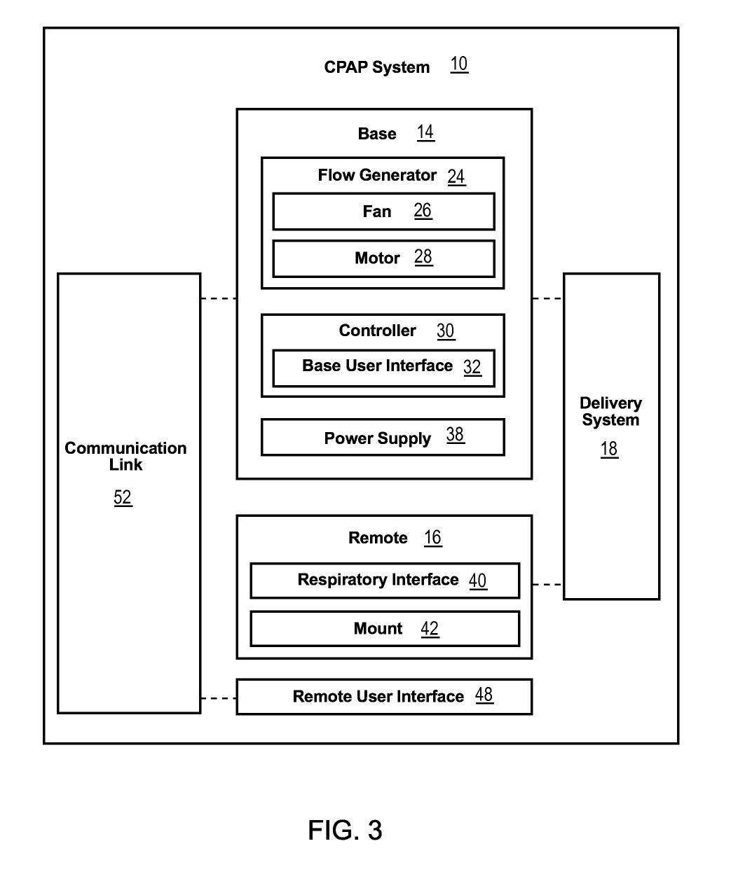

Referring to FIGS. 1-3, in selected embodiments of an apparatus and method in accordance with the present invention, a system 10 may be configured to provide a continuous positive airway pressure (CPAP) therapy to a user 12. In certain embodiments, a system 10 may include a base 14, a remote 16, and a delivery system 18 extending to connect the base 14 to the remote 16. In general, a base 14 may include certain elements or components of a system 10 that are stationary. A remote 16, on the other hand, may include certain elements or components of a system 10 that move with a user 12. A delivery system 18 may connect the base 14 to the remote 16 and accommodate relative motion therebetween.

For example, a system 10 in accordance with the present invention may maintain a positive pressure against which a user 12 breathes during sleep. Accordingly, a system 10 may be configured to be used by the user 12 while he or she is resting in bed. In certain embodiments, a base 14 may be configured to rest on a supporting surface (e.g., a side table 20, floor, or the like, or some combination thereof) on or near the bed of a user 12. A remote 16 may be secured to move with the head of the user 12. Thus, as a user 12 moves (e.g., shifts or rolls over in bed) and a base 14 remains stationary, the distance between the base 14 and the remote 16 may change. A delivery system 18 may have a length and flexibility selected to accommodate such changes in distance and direction.

A base 14 may include a housing 22. A housing 22 may contain or support certain basic elements of a base 14. For example, a housing 22 may contain a flow generator 24. A flow generator 24 may be the prime mover of air within a base 14. A flow generator 24 may draw air from the environment, through a filter, or without a filter, and pressurize it sufficiently to maintain a positive pressure against which a user 12 breathes during sleep.

From a flow generator 24, a delivery system 18 may provide a passageway to carry the air to a remote 16 for delivery into the nostrils, mouth, or both of a user 12. In typical usage, a pressure increase of a fraction of a pound per square inch or a fraction of a kilogram per square centimeter will be provided by the system 10 to the airway of a user 12.

A flow generator 24 in accordance with the present invention may have any suitable configuration. In selected embodiments, a flow generator 24 may comprise a fan 26 and a motor 28. The motor 28 may drive the fan 26. The fan 26 may be a squirrel-cage type fan 26 and the motor 28 may be embedded within the outer envelope (dimensions) or other confines of the fan 26. Alternatively, a motor 28 be positioned outside the confines of a fan 26 and conduct rotation thereto via one or more gears, belts, chains, shafts, other connectors, or the like.

The spinning of the fan 26 may cause air to move radially or axially away from the fan 26. A shroud may surround the fan 26 to direct the air to an output duct to which a delivery system 18 may connect. In certain embodiments, a flow generator 24 (e.g., an inlet to a flow generator 24) may be protected by a grid 29, grill 29, mesh 29, filter 29, guard 29, or the like to prevent entry of fingers, other small objects, particles, or the like into the flow generator 24.

A base 14 may include a controller 30. A controller 30 may be designed to be as simple or sophisticated as desired for the appropriate therapy. At a rudimentary level, a controller 30 may enable a base 14 (e.g., flow generator 24 of a base 14) to be turned “on” and “off.” In a more sophisticated embodiment, a controller 24 may enable a user 12 or medical personnel to select or control the pressure, net air flow, profile of the increase of pressure of the air flow, or the like in order to provide for the comfort and therapy of the user 12.

In selected embodiments, a controller 30 may include a base user interface 32 (i.e., a user interface 32 formed as part of the base 14). A base user interface 32 may provide a mechanism through which a user may issue commands to a controller 30. In certain embodiments, a base user interface 32 may include various buttons 34, switches 34, or the like. For example, one button 34a may be a switch to turn the flow generator 24 on and off. Other buttons 34b, 34c may control an increase or decrease in the speed of a fan 26.

Still other buttons 34d may control other features, including a display 36. A display 36 may communicate instructions or provide feedback information regarding pressure, fan speed, or the like. A display 36 may present interactive selections (e.g., menus or the like) enabling a user 12 to control one or more aspects of a system 10. In general, information and instructions by way of warning and basic set up may also be included in a label simply printed and adhered to a portion of the system 10 (e.g., on the housing 22 of the base 14).

A base 14 may include a power supply 38. A power supply 38 may provide power to drive the flow generator 24. In certain embodiments, a pneumatic power supply 38 may be provided. Alternatively, a power supply 38 may rely on or comprise either wall current or battery power. For example, to provide completely self-contained power, a power supply 38 may comprise a rechargeable battery (e.g., an external battery pack). Alternatively, a power supply 38 may comprise a plug enabling a base 14 to draw power from a wall outlet. In yet another alternative embodiment, a power supply 38 may comprise both in order that a system 10 may be recharged when the wall current is available, but still remain functional when wall current is not available.

In certain embodiments, a power supply 38 may include or comprise an external cord system (e.g., an AC adapter) delivering power from a wall outlet to a plug. The plug may fit into a jack formed within the housing 22, base user interface 32, or the like in order to access the flow generator 24 and power it. In embodiments where a battery is powering the base 14, the cord system may simply operate to power the battery during recharging.

A remote 16 in accordance with the present invention may have any suitable configuration. In selected embodiments, a remote 16 may include a respiratory interface 40. A respiratory interface 40 may be formed in any suitable manner. In general, a respiratory interface 40 may comprise flexible material for contacting and sealing against the face of a user 12. In certain embodiments, a respiratory interface 40 may comprise a mask covering the entire face, only the nostrils, the nostrils and the mouth, or only the mouth of a user 12. Accordingly, a pressurized flow of air generated by a flow generator 24 may be delivered via a delivery system 18 to one or more external respiratory openings of a user 12.

A remote 16 may further include a mount 42. A mount 42 may comprise one or more structures for securing a respiratory interface 40 to the face or head of a user 12. A mount 42 may ensure that a respiratory interface 40 moves with the head of a user 12 and maintains a proper position with respect to the corresponding respiratory openings. In selected embodiments, a mount 42 may include one or more extensions 44 (e.g., rigid extensions, monolithic extensions, or the like) extending from a respiratory interface 40. Alternatively, or in addition thereto, a mount 42 may include one or more straps 46 (e.g., woven, knitted, or solid bands, typically with elastic properties). Such straps 46 may adjustably extend around the head, neck, or some combination thereof to provide a desired securement and sealing (e.g., compression against the skin of the user 12) of the respiratory interface 40.

In that a system 10 may provide treatment as a user 12 sleeps, the control of a user 12 over the operation of the system 10 may be limited by one or more factors. For example, when a user 12 is reclined in bed, a base 14 (or user interface 32 thereof) may not be within reach or easy reach. Additionally, if a user 12 wears corrective lens (e.g., contact lens or glasses), they may be removed as he or she readies for bed. Accordingly, a user 12 may be required to manipulate a system 10 (e.g., don a remote 16, doff a remote 16, turn a flow generator 24 on, turn a flow generator 24 off, or the like) with impaired vision. Furthermore, a user 12 may be required to manipulate a system 10 in poor lighting conditions (e.g., in the dark). Moreover, a user 12 may be required to manipulate a system 10 while not fully awake or alert.

For example, after retiring for the night, a user 12 may receive a telephone call. Should the user 12 be expecting the interruption or otherwise desire to answer the call, he or she may need remove the remote 16. The user 12 may also need to turn off the airflow and the motor of the flow generator 24 to prevent any noise corresponding thereto from interfering with the call. A user 12 may be required to execute these tasks in a hurry, in the dark, without corrective lens, without being fully awake and alert, without having the base user interface 32 in easy reach, and so forth. Accordingly, such tasks may be difficult to perform quickly and efficiently, particularly the task of locating and actuating an “off” button on a base 14.

In view of these and related challenges, a system 10 in accordance with the present invention may include a remote user interface 48 (i.e., a user interface 38 that is spaced from a base 14). In selected embodiments, a remote user interface 48 may provide a mechanism through which a user may issue commands to a controller 30. For example, a remote user interface 48 may include one or more input devices 50 (e.g., one or more buttons 50, switches 50, sensors 50, or the like) for received a command from a user 12 to turn a flow generator 24 on or off. Other commands that may be received by a remote user interface 48 may include changes in air pressure (e.g., speed of fan 26 within a flow generator 24), changes in lighting provided by or corresponding to a base 14, changes in air humidity, or the like.

A remote user interface 48 may comprise a control unit separable from a remote 16. Alternatively, a remote user interface 48 may be integrated within (e.g., be built into) a remote 16 or some hardware moving therewith. In either case, a remote 16 may be connected to move with the head of a user 12. Accordingly, in selected embodiments, regardless of the orientation of the user 12, the remote user interface 48 may be in the same position with respect to the head or face of the user 12. A user 12 may, therefore, quickly and repeatably locate the remote user interface 48 and actuate one or more input devices 50 corresponding thereto. The user 12 may do this in the dark by habit, muscle memory, reflex, or touch alone. Moreover, the user 12 need not sit up, reach for, nor locate a base 14 or base user interface 32.

In selected embodiments, a remote user interface 48 may be secured to something that does not move strictly with the head of a user 12. For example, a remote user interface 48 may be secured to a stationary object (e.g., the head board of a bed at a location easily and predictably reached by a user 12). Alternatively, a remote user interface 48 may be connected to, or integrated within, an object that is not stationary. For example, a remote user interface 48 may be connected to a delivery system 18 spaced at some distance from a remote 16. Accordingly, the remote user interface 48 may move as a delivery system 18 flexes and moves to adjust to changes in the position of the user 12. However, the remote user interface 48 need not always have the same relative positioning with respect to the head of a user 12.

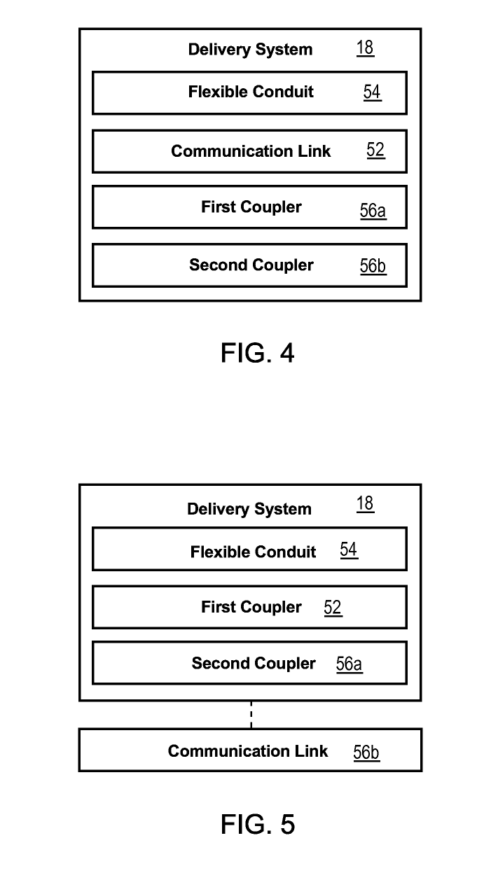

Referring to FIGS. 1, 4, and 5, a remote user interface 48 may communicate or interact with a base 14 in any suitable manner. In selected embodiments, a communication link 52 may communicate to a base 14 (e.g., a controller 30 of a base 14) the commands input at a remote user interface 48. A communication link 52 may be wired or wireless. For example, in selected embodiments, a communication link 52 may comprise wireless communication (e.g., Bluetooth or other radio frequency communication) between a remote user interface 48 and a base 14.

Alternatively, a communication link 52 may comprise a wire connection. For example, in selected embodiments, a delivery system 18 may comprise an elongated flexible conduit 54, a first coupler 56a, and a second coupler 56b. A first coupler 56a may engage one end of a flexible conduit 54 and support engagement with a base 14. A second coupler 56b may engage an opposite end of the flexible conduit 54 and engage a remote 16 or hardware corresponding thereto. Accordingly, a delivery system 18 may form a bridge connecting a remote 16 to a base 14. Thus, in certain embodiments, a communication link 52 may comprise a cable 58 extending along the delivery system 18 to connect the remote user interface 48 with the base 14.

In selected embodiments, a cable 58 may be integrated within a flexible conduit 54. For example, a flexible conduit 54 may be formed as a convoluted hose comprising a coil and barrier material. Barrier material may define the path or tube for the air traveling within a flexible conduit 54. The coil may support the barrier material, enabling the barrier material to bend and flex without collapsing and blocking the flow of air through the flexible conduit 54. A cable 58 may be embedded within one of the coil or barrier material. The cable 58 may extend helically or axially with respect to the flexible conduit 54.

Alternatively, a cable 58 may be a separable unit connected to a flexible conduit 54 to extend and move therewith. For example, a cable 58 (e.g., an insulated wire pair or the like) may be bonded, clipped, tied, wrapped, or otherwise secured to a flexible conduit 54. The cable 58 may extend helically or axially with respect to the flexible conduit 54. Accordingly, as the flexible conduit 54 bends and flexes, the cable 58 may bend and flex with it.

Couplers 56a, 56b in accordance with the present invention may have any suitable configuration. In selected embodiments, couplers 56a, 56b may correspond to a particular standard (e.g., a 22 mm standard). Alternatively, or in addition thereto, couplers 56a, 56b may assist in forming an electrical connection between a remote user interface 48 and a base 14. For example, a first coupler 56a may include a plug. The plug may connect to one end of a cable 58. In such embodiments, an engagement between a coupler 56a and a base 14 may include an engagement between a plug corresponding to the coupler 56a and a receptacle corresponding to the base 14. In certain embodiments, a similar plug may be included within a second coupler 56a. Accordingly, a second coupler 56b may establish an electrical connection between a cable 58 and a remote 16 or some component corresponding to a remote 16. Alternatively, the mechanical coupler 56a may have a set of electrical contacts acting as the second coupler 56b.

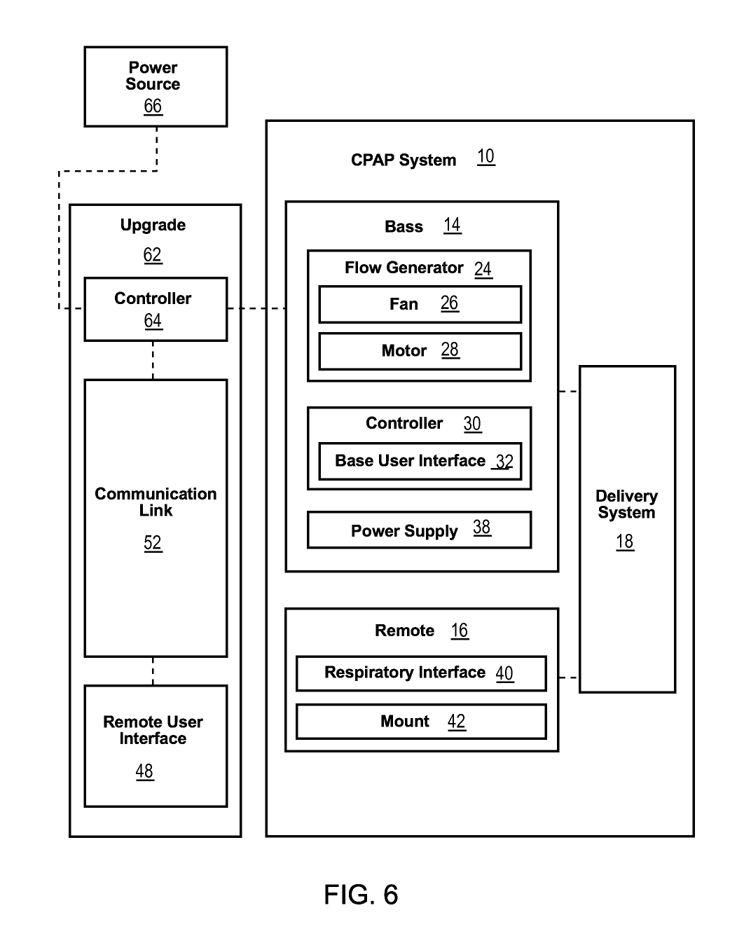

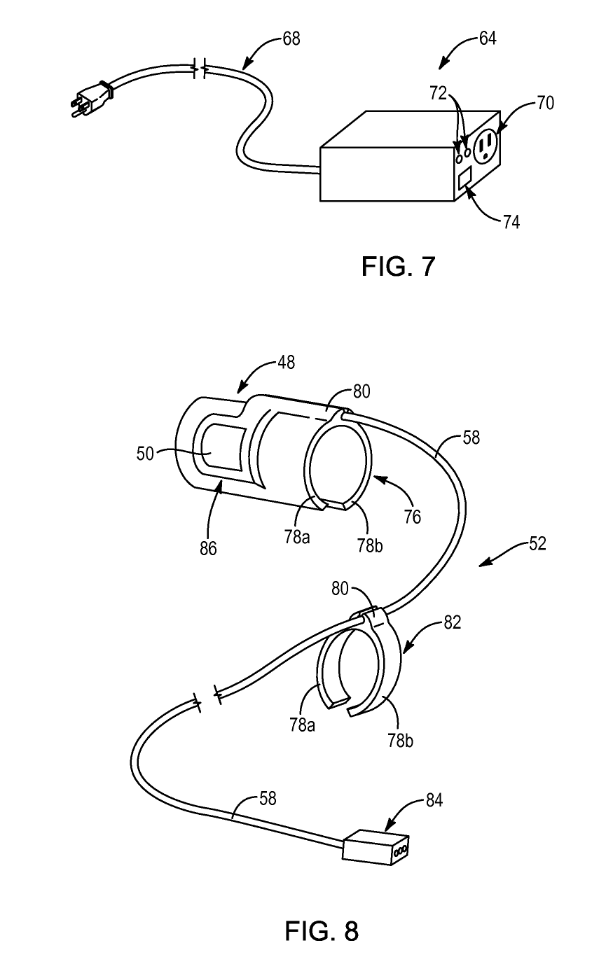

Referring to FIGS. 6 and 7, in selected embodiments, a system 10 in accordance with the present invention may include a base 14 configured to communicate directly with a remote user interface 48. Alternatively, a system 10 may include or make use of a CPAP system lacking such functionality (e.g., a non-communicative CPAP system 60).

For example, in selected embodiments, a user 12 may own or use a non-communicative CPAP system 60. Should the user 12 desire the benefits of a remote user interface 48 in accordance with the present invention, the user 12 may have two options. First the user 12 may obtain a base 14 configured to communicate directly with a remote user interface 48. Alternatively, a user 12 may obtain an upgrade 62. An upgrade 62 may expand the capabilities of a non-communicative system 60 to provide the benefits of a remote user interface 48.

An upgrade 62 may function in any suitable manner. In selected embodiments, an upgrade 62 may include hardware, software, or some combination thereof that modifies a non-communicative system 60. For example, an upgrade 62 may include a replacement for a base user interface 32, an add-on component shaped and sized to fit within a housing 22, or the like. Alternatively, an upgrade 62 may not modify a non-communicative system 60, but simply externally control the operation thereof.

For example, in selected embodiments, an upgrade 62 may comprise a controller 64, remote user interface 48, and communication link 52. The communication link 52 may enable a remote user interface 48 to issue one or more commands to the controller 64. A controller 64 may be positioned and connected to control the flow of power (e.g., electrical power) to a base 14. Accordingly, when instructed by a user 12 via a remote user interface 48, a controller 64 may cut the power to a base 14, effectively turning a corresponding flow generator 24 off. Conversely, when instructed by a user 12 via a remote user interface 48, a controller 64 may reapply the power to a base 14, effectively turning the corresponding flow generator 24 on.

A controller 64 may control the flow of power to a base 14 in any suitable manner. In selected embodiments, a controller 64 may be inserted between a power source 66 (e.g., an electrical outlet) and a power supply 38 of a base 14. For example, a controller 64 may include a power cord 68 and a receptacle 70. The power cord 68 may be connected to an electrical outlet. The receptacle 70 may receive a plug corresponding to a power supply 38 (i.e., the plug that would otherwise be inserted within the electrical outlet directly). Accordingly, a controller 64 may control whatever portion of the power received from the electrical outlet is passed on to the power supply 38 of a base 14.

In selected embodiments, a power supply 38 may include one or more batteries. For example, a power supply 38 may include an external battery enabling a CPAP system 60 to continue operating in the event of a power outage. In such embodiments, a controller 64 may be inserted between the external battery and the corresponding base 14. A controller 64 may include one or more jacks 72 to enable such a connection scheme. A controller 64 may also include one or more power conditioning functions (e.g., AC/DC conversion or the like) as desired or necessary.

A controller 64 in accordance with the present invention may communicate with a remote user interface 48 in any suitable manner. As noted above, a communication link 52 may support wireless or wired communication. Accordingly, a controller 64 may include or operate in conjunction with a wireless receiver. Alternatively, a controller 64 may include or operate in conjunction with a receptacle 74 configured to connect with one end of a cable 58 extending from a remote user interface 48.

Referring to FIG. 7, a remote user interface 48 may connect to a remote 16 (e.g., respiratory interface 40, mount 42, extension 44, strap 46, or the like), delivery system 18 (e.g., flexible conduit 54, second coupler 56b, or the like), or some other object (e.g., bed headboard, wall, side table, or the like) in any suitable manner. Suitable connection schemes may include or employ integrated formation (e.g., building a remote user interface 48 into some other component like a remote 16, delivery system 18, or the like), clips, clamps, straps, bands (e.g., bands exhibiting certain elastic properties), snaps, adhesive, hook-and-loop engagements, screws, bolts, or the like or combinations or sub-combinations thereof.

For example, in selected embodiments, a remote user interface 48 may include or be connected to a mount 76 (e.g., clip, clamp, band, strap, etc.) sized and shaped to engage a delivery system 18 or some portion thereof (e.g., a coupler 56a, 56b, flexible conduit 54, some combination thereof, or the like). A mount 76 may comprise a material and shape exhibiting sufficient flexibility, elasticity, or some combination thereof to enable it to be applied to or engage a delivery system 18.

In certain embodiments, a mount 76 may comprise opposing first and second extensions 78a, 78b. The extensions 78a, 78b may be flexed (e.g., resiliently flexed) apart to admit a portion of delivery system 18 therebetween. Once released, the extensions 78a, 78b may grip the delivery system 18 and secure the remote user interface 48 with respect to thereto.

As with other remote user interfaces 48, a remote user interface 48 corresponding to a mount 76 may communicate with a base 14, controller 64, or the like using wired or wireless communication. In wired embodiments, a mount 76 may support a connection between a cable 58 and one or more input devices 50 of a remote user interface 48. For example, a mount 76 may include or support a receptacle for receiving a plug connected to one end of a cable 58. Alternatively, as shown in the illustrated embodiment, a mount 76 may include a wire support 80 for directly securing or supporting a cable 58.

In selected embodiments, one or more mounts 82 may be distributed along a delivery system 18 (e.g., flexible conduit 54) to assist in connecting a cable 58 to the delivery system 18, thereby ensuring that the cable 58 moves and bends therewithin Like a mount 76 corresponding to a remote user interface 48, a mount 82 for securing a cable 58 may be sized and shaped to engage a delivery system 18 or some portion thereof. In certain embodiments, a mount 82 may comprise a wire support 80 for directly securing or supporting a cable 58.

Additionally, a mount 82 may include opposing first and second extensions 78a, 78b that may be flexed apart to admit a portion of delivery system 18 therebetween. Once released, the extensions 78a, 78b may grip the delivery system 18 and secure the cable 58 with respect thereto. One end of a cable 58 may be free to extend and engage a base 14, controller 64 or the like. For example, in selected embodiments, the end of a cable 58 opposite a remote user interface 48 may include or connect to a plug 84 sized and shaped to engage a corresponding receptacle 74 on a controller 64, or vice versa.

It may be undesirable, unhealthy, or potentially dangerous for a user to inadvertently actuate or activate an input device 50. That is, a system 10 in accordance with the present invention may provide treatment while a user 12 is asleep. If an input device 50 were engaged while the user 12 is asleep, the user 12 may not immediately realize that the important or necessary treatment has been altered, terminated, or the like.

Accordingly, in selected embodiments, a remote user interface 48 may be configured to prevent or resist inadvertent actuation or activation of an input device 50. This may be accomplished in any suitable manner. In certain embodiments, one or more protections may be included to ensure that something more than unintentional contact (e.g., bumps or pressure resulting from rolling over, contact with a pillow, contact with an arm, or the like) is required for actuation or activation of an input device 50.

For example, access to one or more input devices 50 may be protected or limited by a shroud, cover, border 86, flush or sub-surface mounting of the one or more input devices 50, or the like. Alternatively, or in addition thereto, proper actuation or activation of one or more input devices 50 may require some motion inherently absent in unintentional contact. For example, one or more input devices 50 may require a deliberate sliding motion, rotating motion, pushing, pulling, or the like, or some combination thereof. The particular motion, the force required to execute it, or both may be selected to render de minimis the possibility of inadvertent actuation.

In selected embodiments, two or more input devices 50 may require simultaneous actuation. For example, a remote user interface 48 may include two input devices 50 (e.g., two buttons 50). A first such input device 50 may be located at a first location (e.g., on a first extension 78a). A second input device 50 may be located at a second location (e.g., on a second extension 78b) spaced from the first location. For proper actuation, it may be required that both the first and second input devices 50 be actuated (e.g., depressed) simultaneously. Due to the relative positioning of the first and second input devices 50, the possibility of inadvertent, simultaneous actuation thereof may be very low.

For example, the first and second input devices 50 may be positioned on opposite sides of a mechanical component (e.g., a mount 76, delivery system 18, respirator interface 40, or the like). This makes highly unlikely any inadvertent bumps or pressure resulting from rolling over, contact with a pillow, or the like causing simultaneous actuation.

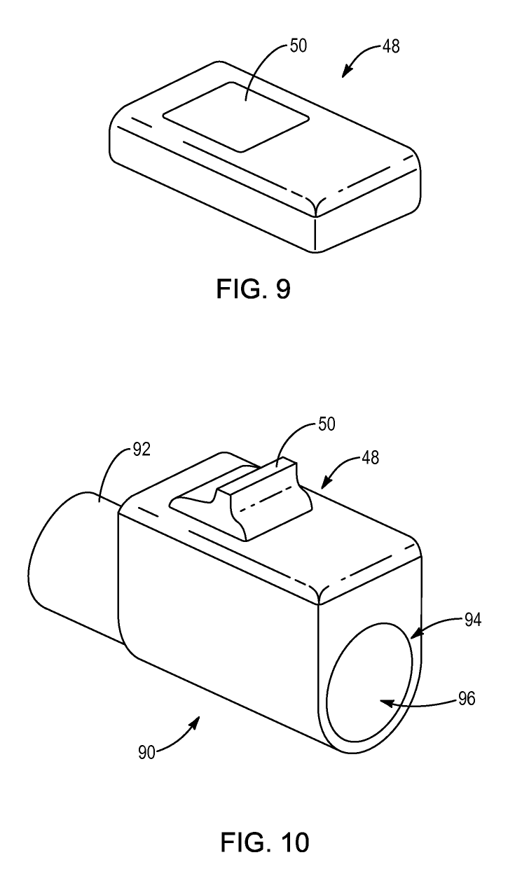

Referring to FIG. 9, different users 12 may prefer different locations for their remote user interfaces 48. In selected embodiments, a remote user interface 48 may be placed or secured at any location selected or desired by a user 12. This flexibility may be facilitated by providing a user interface 48 within a small, unobtrusive housing 88. While not required, it may be further facilitated by a communication link 52 that is wireless. Moreover, a remote user interface 48 may be sold or supplied with one or more securement mechanisms supporting attachment of the housing 88 to a variety of locations.

For example, a remote user interface 48 may be supplied with one or more clips, straps, bands, snaps, adhesives (e.g., adhesive patches), hook-and-loop engagements, screws, bolts, or the like. Such securement mechanisms may enable a user 12 to personally select and change the location at which he or she would like to place or secure a remote user interface 48. Accordingly, a user 12 may secure (e.g., removably secure) a housing 88 to a stationary object (e.g., headboard, wall, side table, or the like) or a movable object (e.g., an article of clothing, hair, a body part, bedding, a remote 16, a delivery system 18, or the like).

For example, one or more securement mechanisms may enable a user 12 to wear a housing 88 on a finger, hand, wrist, arm, or the like. One or more securement mechanisms may enable a user 12 to secure a housing 88 on a respiratory interface 40, strap 46, extension 48, coupler 56a, 56b, flexible conduit 54, or the like. For example, in certain embodiments, one or more straps 56 may have an exterior provided with either hook or loop material. Accordingly, the corresponding loop or hook material extending from a housing 88 (e.g., a back side of a housing 88) may enable a user 12 to secure that housing 88 anywhere along the straps 56. Thus, a user 12 may have significant flexibility in selecting where to locate a remote user interface 48.

Referring to FIG. 10, in selected embodiments, a remote user interface 48 may be integrally formed with or built into a remote 16, delivery system 18, or component thereof. Such embodiments may provide simplicity and elegance in function and appearance. In certain embodiments, a remote user interface 48 may be built into a wide range of different remotes 16, delivery systems 18, or the like. Accordingly, a user 12 may enjoy the benefits of a remote user interface 48, while still being free to choose a respiratory interface 40, delivery system 18, or the like best matching the personal preferences of the user 12.

Alternatively, a remote user interface 48 may be built into fewer articles of more universal usage or application. For example, a remote user interface 48 may be built into a limited set of articles that may be employed by a wide range of users 12, regardless of the particular style, function, brand, etc. of the remote 16, delivery system 18, or the like being used or preferred. For example, in selected embodiments, a remote user interface 48 may be built into an insert 90 or adapter 90.

In certain embodiments, an insert 90 may be inserted between a coupler 56b and a remote 16. The insert 90 may include an extension 92 (e.g., extending to engaging a remote 16) and an aperture 94 (e.g., receiving an extension of a coupler 56b). A through hole 96 may extend axially through the insert 90. By accommodating certain common or standard sizes (e.g., diameters) of extensions and apertures, very few models of inserts 90 may be needed to bring the benefits of a remote user interface 48 to a wide variety of remotes 16, delivery systems 18, and combinations thereof.

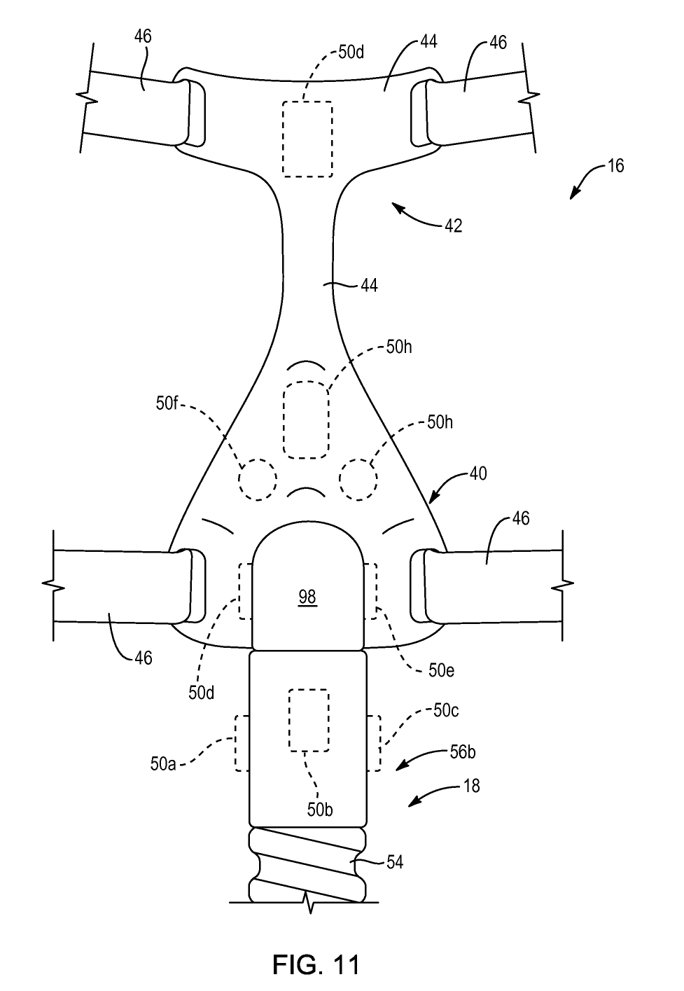

Referring to FIG. 11, a remote user interface 48 (e.g., one or more input devices 50) may be built in or connected to a wide variety of locations on a remote 16, delivery system 18, or the like. The locations may vary according to the shapes and sizes of the various components. For example, different locations may be selected, based on whether the respiratory interface 40 covers just the nostrils, the nostrils and mouth, or just the mouth.

In certain embodiments, one or more input devices 50a, 50b, 50c may be built in or connected to a coupler 56b. Alternatively, one or more input devices 50d, 50e may be built into or connected to a pivot 98. A pivot 98 may be formed as part of or connected to a respiratory interface 40 and enable a coupler 56b to pivot with respect to the respiratory interface 40. In other embodiments, one or more input devices 50f, 50g, 50h may be built into or connected to a respiratory interface 40. In still other embodiments, one or more input devices 50i may be built in or connected to a mount 42, extension 44, strap 46, or the like.

In selected embodiments, certain input device 50 may be configured as pairs. Proper use of such devices 50 may require simultaneous actuation of both. Such pairs may include input devices 50a, 50c positioned on opposites sides of a coupler 56b, input devices 50d, 50e positioned on opposites sides of a pivot 98, input devices 50f, 50g positioned on opposites sides or faces of a respiratory interface 40, or the like.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A CPAP apparatus comprising:
a base comprising a flow generator;
a remote comprising a respiratory interface;
an elongated flexible conduit extending to connect the base to the respiratory interface;
a remote user interface connected to the remote to move therewith; and
the remote user interface selectively transitioning, in accordance with commands input therethrough, the flow generator between an active, “on” condition and an inactive, “off” condition.

2. The apparatus of claim 1, wherein the respiratory interface comprises a mask configured to cover at least one external respiratory opening of a user.

3. The apparatus of claim 2, further comprising a communication link extending to connect the base to the remote user interface.

4. The apparatus of claim 3, wherein the communication link comprises a wired connection between the base and the remote user interface.

5. The apparatus of claim 3, wherein the communication link comprises a wireless connection between the base and the remote user interface.

6. The apparatus of claim 5, wherein the remote user interface comprises a switch.

7. The apparatus of claim 5, wherein the remote user interface comprises a plurality of actuators that must be actuated simultaneously to transition the flow generator between the active and the inactive condition.

8. The apparatus of claim 5, wherein the elongated flexible conduit comprises a first end engaging the base and a second end engaging the respiratory interface.

9. The apparatus of claim 8, wherein the remote further comprises a mount configured to secure the respiratory interface against at least a portion of a face of a user.

10. The apparatus of claim 9, wherein the remote user interface secures directly to at least one of the mount, the respiratory interface, and the second end of the elongated flexible conduit.

11. A system comprising:
a base supported on a surface that is stationary and inanimate, the base comprising a flow generator;
a respiratory interface spaced from the base and positioned in direct fluid communication with at least one respiratory opening of a user;
an elongated flexible conduit extending to connect the base to the respiratory interface and place the flow generator in fluid communication with the respiratory opening;
a remote user interface spaced from, and movable with respect to, the base;
a communication link connecting the base and the remote user interface; and
the remote user interface receiving commands from the user and selectively transitioning, via the communication link in accordance with the commands, the flow generator between an active, “on” condition and an inactive, “off” condition.

12. The apparatus of claim 11, wherein the communication link comprises a wired connection between the base and the remote user interface.

13. The apparatus of claim 11, wherein the communication link comprises a wireless connection between the base and the remote user interface.

14. The apparatus of claim 11, wherein the remote user interface comprises a switch.

15. The apparatus of claim 11, wherein the remote user interface comprises a plurality of actuators that must be actuated simultaneously to transition the flow generator between the active and the inactive condition.

16. The apparatus of claim 11, wherein the elongated flexible conduit comprises a first end engaging the base and a second end engaging the respiratory interface.

17. The apparatus of claim 16, further comprising a mount configured to secure the respiratory interface against at least a portion of a face of a user.

18. The apparatus of claim 17, wherein the remote user interface is secured directly to at least one of the mount, the respiratory interface, and the second end of the elongated flexible conduit.

19. A method comprising:
selecting a CPAP system comprising
a flow generator,
a respiratory interface,
an elongated flexible conduit extending to connect the flow generator to the respiratory interface, and
a remote user interface connected to the respirator interface;
securing, by a user, the remote user interface at a location selected by the user;
positioning the respiratory interface in direct fluid communication with at least one external respiratory opening of the user;
transitioning the flow generator to an active, “on” condition;
breathing, by the user via the at least one external respiratory opening, air pressurized by the flow generator;
actuating, by the user, the remote user interface to transition the flow generator to an inactive, “off” condition.

20. The method of claim 19, further comprising:
resting, by the flow generator during the entirety of the breathing, stationary; and
moving, by the remote user interface during the entirety of the breathing, with the head of the user.
